(19) United States
(12) Patent Application Publication
(10) Pub. No.: US 2020/0346030 A1
(43) Pub. Date: Nov. 5, 2020

(54) COMB WITH A LASER FUNCTION

(71) Applicant: LECC TECHNOLOGY CO., LTD., Taoyuan City (TW)

(72) Inventor: HSIN-CHIH TUNG, KINMEN COUNTY (TW)

(21) Appl. No.: 16/517,867

(22) Filed: Jul. 22, 2019

(30) Foreign Application Priority Data
May 3, 2019 (TW) 108205492

(51) Int. Cl. A61N 5/06; A61H 7/00; A61H 23/00; A46B 15/00; A46B 13/02; A46B 5/00

(52) U.S. Cl. CPC A61N 5/0617; A61N 2005/067; A61H 23/00; A46B 15/0059; A46B 13/023; A46B 15/0034; A46B 15/0004; A46B 15/0024; A46B 5/0041; A46B 15/0018; A46B 15/004; A46B 15/0008; A61H 2201/0173; A61H 2201/10; A61H 2201/5025; A61H 2201/5043; A61H 2205/021; A61H 7/005

(57) ABSTRACT

A comb with a laser function includes a main body, a laser irradiation portion, a vibration portion and a controlling portion. The laser irradiation portion, the vibration portion and the controlling portion are disposed on the main body. The controlling portion is connected to the laser irradiation portion and the vibration portion. The main body includes a brush portion and a handle portion. The brush portion includes a comb tooth portion, and the comb tooth portion includes multiple comb teeth. The laser irradiation portion is adjacent to the comb tooth portion and irradiates a laser beam to a scalp according to the control of the controlling portion. The vibration portion is disposed on the brush portion to generate a vibration according to the control of the controlling portion. The comb with the laser function is capable of irradiating the laser beam to the scalp and massaging the scalp.

### FIELD OF THE DISCLOSURE

The present disclosure relates to a comb, and more particularly to a comb capable of irradiating a laser beam.

### BACKGROUND OF THE DISCLOSURE

An existing comb structure not only provides a convenient way to comb the hair, but is also designed to raise people's willingness to purchase. The primary concern for consumers is whether the combing operation is smooth and satisfactory, which is affected by the number, thickness, length and arrangement of the comb teeth of comb. In addition, when combing the hair with the comb, the comb teeth of the comb also provide a scalp massaging effect. Especially for those who are prone to hair loss or have thin hair, use of the existing comb simply does not meet the need of effectively promoting hair growth.

### SUMMARY OF THE DISCLOSURE

An exemplary embodiment of the present disclosure provides a comb with a laser function, which outputs a laser beam to promote hair growth when combing the hair.

An exemplary embodiment of the present disclosure provides a comb with a laser function including a main body, a laser irradiation portion, a vibration portion and a controlling portion. The main body includes a brush portion and a handle portion. The brush portion includes a comb tooth portion, and the comb tooth portion includes multiple comb teeth. The laser irradiation portion is disposed on the brush portion and is adjacent to the comb tooth portion, and the laser irradiation portion irradiates a laser beam to a person's scalp. The vibration portion is disposed on the brush portion. The controlling portion is disposed on the main body and electrically connected to the laser irradiation portion and the vibration portion to start up the operations of the laser irradiation portion and the vibration portion according.

In summary, the comb with the laser function provided by the exemplary embodiment of the present disclosure outputs the laser beam through the laser irradiation portion to irradiate hair follicles of the scalp and provides a massage effect on the scalp cooperatively through the vibration portion to promote hair growth when combing the hair.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The present disclosure will become more fully understood from the following detailed description and accompanying drawings.

FIG. 1 is a schematic diagram showing a comb with a laser function depicted in accordance with an exemplary embodiment of the present disclosure.

FIG. 2 is a function block diagram of the comb with the laser function depicted in FIG. 1.

FIG. 3 is a schematic diagram showing the comb with the laser function in use depicted in accordance with an exemplary embodiment of the present disclosure.

FIG. 4 is a schematic diagram showing the comb with the laser function depicted in accordance with another exemplary embodiment of the present disclosure.

FIG. 5 is a charging schematic diagram of a comb connected to a charging stand depicted in accordance with an exemplary embodiment of the present disclosure.

FIG. 6 is a charging schematic diagram of a comb connected to another charger depicted in accordance with another exemplary embodiment of the present disclosure.

FIG. 7 is a function block diagram of the comb with the laser function depicted in FIG. 4.

FIG. 8 is a schematic diagram showing the comb with the laser function depicted in accordance with another exemplary embodiment of the present disclosure.

FIG. 9 is a display schematic diagram of a display portion.

FIG. 10 is a function block diagram of the comb with the laser function depicted in FIG. 8.

### DETAILED DESCRIPTION OF THE EXEMPLARY EMBODIMENTS

The present disclosure is more particularly described in the following examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a”, “an”, and “the” includes plural reference, and the meaning of “in” includes “in” and “on”. Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein. Numbering terms such as “first”, “second” or “third” can be used to describe various components, signals or the like, which are for distinguishing one component/signal from another one only, and are not intended to, nor should be construed to impose any substantive limitations on the components, signals or the like.

An exemplary embodiment of the present disclosure provides a comb with a laser function to irradiate the laser beam to the scalp and provide a massage on the scalp when combing the hair of a user with the comb, and have a thin and light structural design so as to not take up too much space, so that a user can carry it easily.

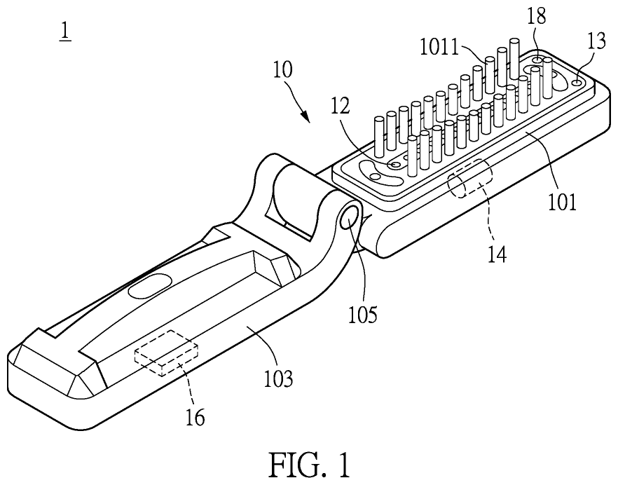

Reference is made to FIG. 1. FIG. 1 is a schematic diagram showing the comb with the laser function depicted in accordance with an exemplary embodiment of the present disclosure. The comb 1 described in the embodiment includes but is not limited to include a main body 10, a laser irradiation portion 12, a vibration portion 14, a controlling portion 16, a static electricity protection portion 18 and a distance detection portion 13. The laser irradiation portion 12, the vibration portion 14, the controlling portion 16, the static electricity protection portion 18 and the distance detection portion 13 are disposed at appropriate positions on the main body 10 according to corresponding functions and designs thereof.

For example, the main body 10 described in the embodiment further includes a brush portion 101, a handle portion 103 and a pivot portion 105. The pivot portion 105 is connected between the brush portion 101 and the handle portion 103, so that the brush portion 101 and the handle portion 103 are foldable against each other to reduce a storage length of the main body 10 and to allow convenient storage thereof. In addition, the brush portion 101 includes a comb tooth portion 1011, and the comb tooth portion 1011 includes multiple comb teeth for combing the hair. The handle portion 105 is held by a person when combing the hair. In an embodiment, the controlling portion 16 is disposed inside the handle portion 105. The laser irradiation portion 12, the vibration portion 14, the static electricity protection portion 18 and the distance detection portion 13 are disposed on the brush portion 101.

The laser irradiation portion 12 provides a function of outputting laser beam herein. The laser irradiation portion 12 further includes a plurality of laser emitters. In an embodiment, the plurality of laser emitters are surface-adhesive laser diodes to reduce a thickness of the brush portion 101 on which the laser irradiation portion 12 is disposed, so that the main body 10 can be lighter and thinner. In addition, a direction in which the laser irradiation potion 12 outputs the laser beam is the same as a direction in which the comb teeth comb the hair. A position at which the laser irradiation portion 12 is disposed is adjacent to the comb tooth portion 1011. For example, several comb teeth of the comb tooth portion 1011 can be arranged in multiple columns. In an embodiment, the laser irradiation portion 12 is disposed at a position between two adjacent rows of the comb teeth. This arrangement can ensure that the laser beam 121 outputted by the laser irradiation portion 12 is irradiated accurately on the scalp when combing the hair with the brush portion 101. The position at which the laser irradiation portion 12 is disposed is not limited to a structure of FIG. 3, and can be disposed at other positions on the brush portion 101.

The vibration portion 14 is disposed inside the brush portion 101 and provides a vibration function for operating, such that the brush portion 101 provides a massage function on the scalp at the same time when combing the hair with the comb 1. For example, the vibration portion 14 can be a vibration motor or an ultrasonic vibrator.

The distance detection portion 13 is used to detect a distance between the brush portion 101 and an object. The laser irradiation portion 12 determines whether to irradiate the laser beam or not according to the detection result. For example, when the distance detection portion 13 detects that the distance between the comb and the object is smaller than a predetermined distance, the laser irradiation portion 12 is allowed to start up. The predetermined distance can be, for example, a distance when the comb is in use and in contact with the hair. Conversely, when the distance detection portion 13 detects that the distance between the comb and the object is larger than the predetermined distance, the laser irradiation portion 12 is not allowed to start up. In addition, in an embodiment, the distance detection portion 13 is adjacently disposed on a side of the laser irradiation portion 12. A direction in which the distance detection portion 13 detects a target is the same as a direction in which the laser irradiation portion 12 outputs the laser beam. In an embodiment, the distance detection portion 13 can be, for example, a proximity sensor or an ultrasonic sensor, and the present disclosure is not limited therein.

The static electricity protection portion 18 is started up to provide a function of a static electricity protection, so as to reduce the static electricity generated from the hair effectively when combing the hair with the brush portion 101. In an embodiment, the static electricity protection portion 18 is a negative ion irradiation circuit or a contact static electricity protection circuit, but the present disclosure is not limited therein.

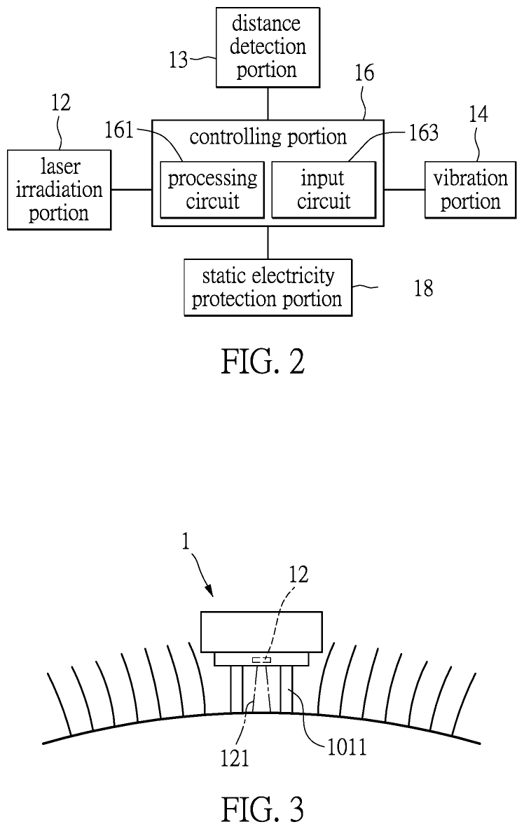

Reference is made to FIG. 2. FIG. 2 is a function block diagram of the comb with the laser function depicted in accordance with an exemplary embodiment of the present disclosure. The controlling portion 16 depicted in FIG. 1 refers to other operations than the combing function provided by the person turning on or off the comb. The controlling portion 16 depicted in FIG. 2 is electrically connected to the laser irradiation portion 12, the distance detection portion 13, the vibration portion 14 and the static electricity protection portion 18. Therefore, the controlling portion 16 can turn on or off the laser irradiation portion 12, the distance detection portion 13, the vibration portion 14 and the static electricity protection portion 18 for operating functions.

More specifically, the controlling portion 16 includes a processing circuit 161 and an input circuit 163. The processing circuit 161 is electrically connected to the input circuit 163. The processing circuit 161 can be, for example, related to a hardware circuit having calculating function, and can be one or any combination of an application specific integrated circuit (ASIC), a field programmable gate array (FPGA), or a system on a chip (SOC). The input circuit 163 can be used to input an input signal through a human interface by a person for operating the comb. The input circuit 163 can be, for example, a switch interface or a touch interface. Therefore, the processing portion 161 can control the laser irradiation portion 12, the distance detection portion 13, the vibration portion 14 and the static electricity protection portion 18 correspondingly according to the input signal inputted from the input circuit 163.

For instance, a user turns on one or more functions of the laser irradiation portion 12, the distance detection portion 13, the vibration portion 14 and the static electricity protection portion 18 according to demands through operations of the input circuit 163. Similarly, the user can turn off the laser irradiation portion 12, the distance detection portion 13, the vibration portion 14 and the static electricity protection portion 18 through operations of the input circuit 163.

In an embodiment, when the input signal inputted from the input circuit 163 is a first input signal, the processing circuit 161 starts up the laser irradiation portion 12; when the input signal is a second input signal, the processing circuit 16 starts up the laser irradiation portion 12 and the vibration portion 14. In an embodiment, when the laser irradiation portion 12 and the vibration portion 14 are turned on, the vibration portion 14 can continuously generate vibration or intermittent vibration.

More specifically, in an example of the comb 1 depicted in the embodiment, when the controlling portion 16 starts up the laser irradiation portion 12 and the user holds the comb 1 to comb the hair as shown in FIG. 3, the laser irradiation portion 12 irradiates the laser beam to a fixed position of the scalp for a period of time. The controlling portion 12 can output a prompt signal to prompt the user to move the comb to the next position of the scalp after expiration of the period of time. The prompt signal herein may be a vibration signal outputted from the vibration portion 14. The above-described manner is repeatedly performed until the laser irradiation portion 12 is turned off. Through such operation guidance, the person can effectively use the laser irradiation function provided by the comb 1 to appropriately and safely irradiate the positions of the scalp.

It should be noted that in the comb 1 structure depicted in FIG. 1, the distance detection portion 13 and the static electricity protection portion 18 are elastically set according to the actual needs and the product functions. For example, when the comb 1 depicted in FIG. 1 is not provided with the distance detection portion 13 in another embodiment and the laser irradiation portion 12 is activated, the laser emitters can immediately output laser beams.

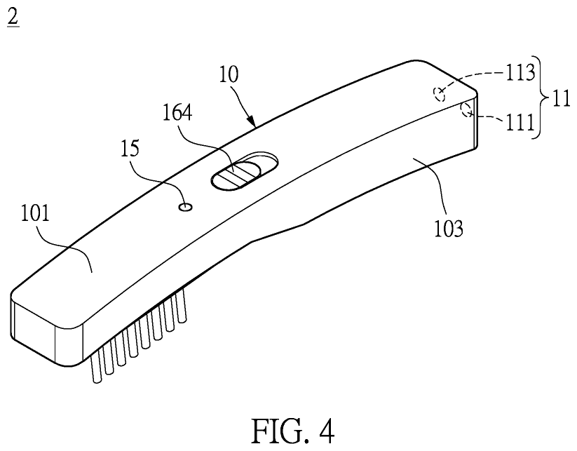

Reference is made to FIG. 4. FIG. 4 is a schematic diagram showing the comb with the laser function depicted in accordance with another exemplary embodiment of the present disclosure. Compared with the comb 1 depicted in FIG. 1, the main body 10 of the comb 2 depicted in the embodiment is not designed with a foldable structure, and the brush portion 101 and the handle portion 103 are directly connected to each other. The main body 10 has a built-in rechargeable battery module (not shown in figure). The rechargeable battery module of the comb 2 can be charged through a charging portion 11 at an end of the main body 10. In an embodiment, the charging portion can be a plurality of different charging interfaces designed as the charging jack 111 and the charging port 113, and the present disclosure is not limited therein.

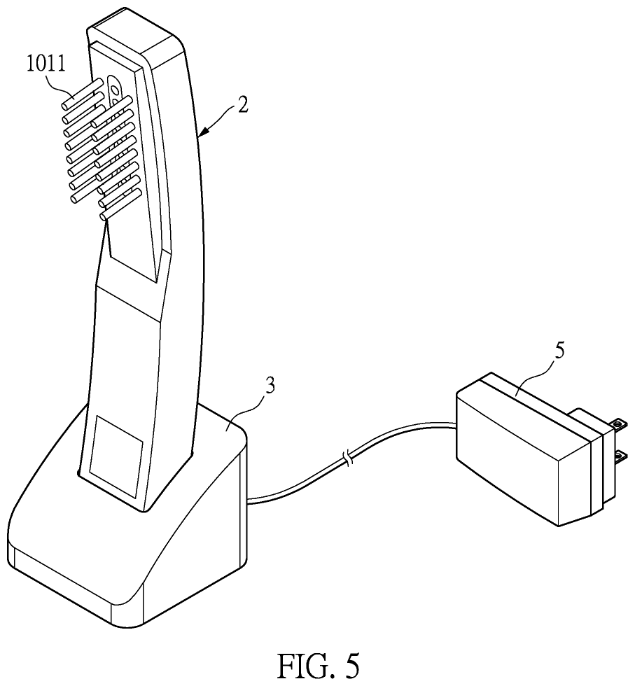

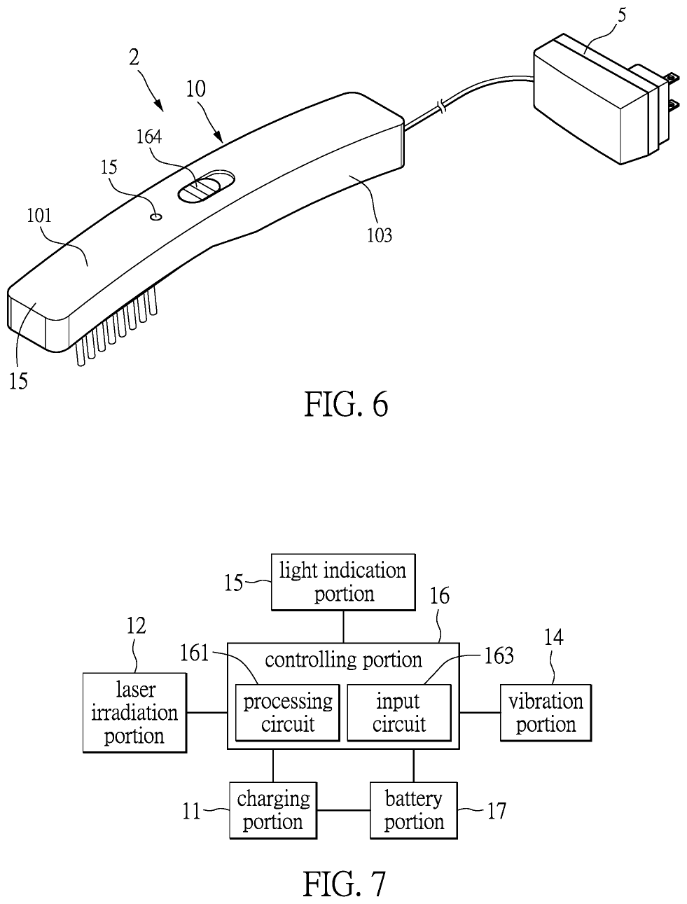

As shown in FIG. 5, the comb 2 is charged through a charger stand 3. The comb is placed in the charging stand 3, and the charging stand 3 is electrically connected to the charger 5. When the charger 5 is plugged into the mains socket, the charging port 113 of the comb 2 at this time will electrically contact the charging interface at the bottom of the charging stand 3, and the comb is charged by the charging stand 3. As shown in FIG. 6, the charging jack 111 of the comb 2 is externally connected to the charger 5, and is then plugged into the mains socket through the charger 5, so that the comb 2 can be charged by the charger 5.

Reference is made to FIG. 7. FIG. 7 is a function block diagram of the comb with the laser function depicted in FIG. 4. The controlling portion 16 depicted in FIG. 7 is electrically connected to the charging portion 11, the laser irradiation portion 12, the vibration portion 14, a light indication portion 15 and a battery portion. The functional operations of the laser irradiation portion 12 and the vibration portion 14 controlled by the controlling portion 16 are the same as the above description, and therefore will not be described herein. In this embodiment, the controlling portion 16 can also control the charging portion 11 to charge the battery portion 17, and control the light indication portion 15 to output different light indication signals by different colors or different blinking variations to represent the current charging state of the battery portion, the electricity of the battery portion or the relevant working state of comb 2.

The input circuit 163 depicted in FIG. 4 is exemplified by a dip switch 164. For example, the dip switch 164 can provide three different positions for sliding switch. The first position can be used as a power switch, the second position can be used as a switch of the laser and vibration functions, and the third position can be used as a switch of the laser function, and the switch control manner of the present disclosure is not limited herein.

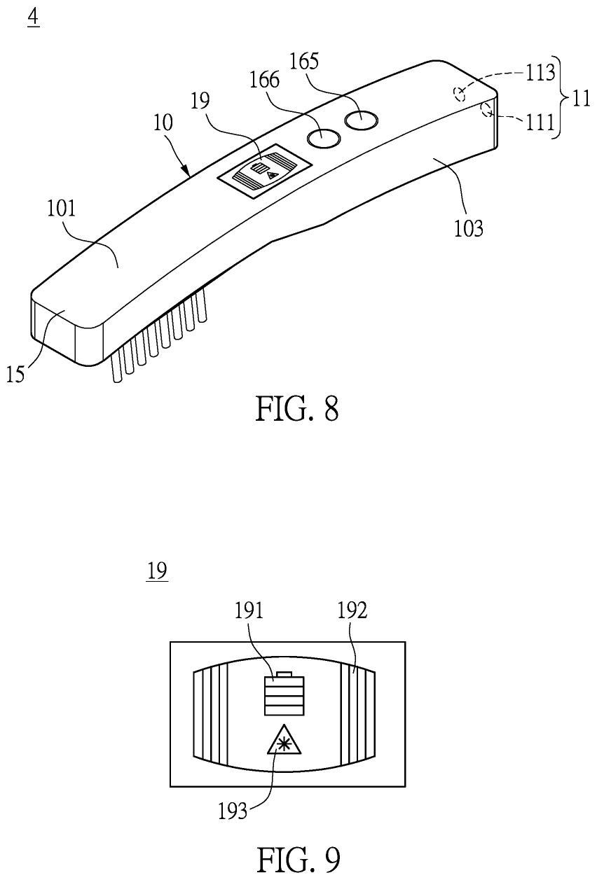

Reference is made to FIG. 8. FIG. 8 is a schematic diagram showing the comb with the laser function depicted in accordance with another exemplary embodiment of the present disclosure. Compared with the comb 2 depicted in FIG. 4, the comb 4 depicted in the embodiment includes a display portion 19, and the input circuit 163 is implemented by a push-button switch 165 and a push-button switch 166. The display portion 19 displays the relevant state of the comb 4. As shown in FIG. 9, the display portion 19 can correspondingly display an operation message according to the control of the control portion 16. The operation message includes, for example, at least a power state 191 of the comb 4, a using time 192 of the comb 4, or a working state 193 of the laser irradiation portion 12, and the present disclosure is not limited therein. The above-described using time 192 refers to the using time after the laser irradiation portion 12 is activated

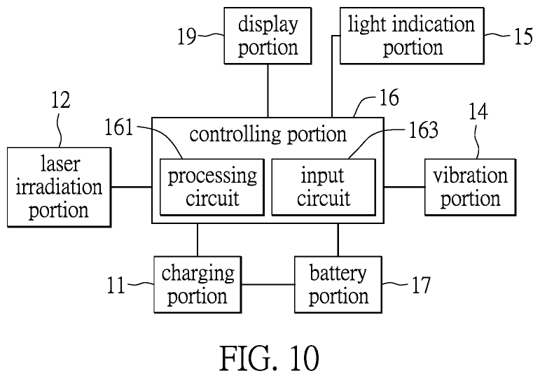

Reference is made to FIG. 10. FIG. 10 is a function block diagram of the comb with the laser function depicted in FIG. 8. The controlling portion 16 depicted in FIG. 10 is electrically connected to the charging portion 11, the laser irradiation portion 12, the vibration portion 14, a light indication portion 15, the battery portion 17, and the display portion 19. The functional operations of the laser irradiation portion 12 and the vibration portion 14 controlled by the controlling portion 16 are the same as the above description, and therefore will not be described herein. In this embodiment, the controlling portion 16 can also control the display portion 19 to display the relevant state of the comb 4.

The input circuit 163 depicted in FIG. 8 is exemplified by the push-button switch 165 and the push-button switch 166. The push-button switch 165 can function as a power button and the push-button switch 166 can function as a laser source button. For example, when the push-button switch 165 is pressed for a predetermined time to turn on the power, and then when the push-button switch 166 is pressed, the laser irradiation portion 12 and the vibration portion 14 can be turned on. In an embodiment, a buzzer (not shown in figure) in the comb 4 can be turned on to generate an audio signal to be used as the aforementioned prompt signal. When the push-button switch 166 is pressed again, the function of the vibration portion 14 and the buzzer can be turned off. If the laser irradiation is to be stopped, the button switch 165 should be pressed again for a while to turn off the power.

[Advantages of the Embodiment]

The comb with a laser function provided by the exemplary embodiment of the present disclosure outputs the laser beam through the laser irradiation portion to irradiate hair follicles of the scalp and provides a massaging effect on the scalp cooperatively through the vibration portion to promote hair growth when combing the hair.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. A comb with a laser function, comprising:
a main body including a brush portion and a handle portion, wherein the brush portion includes a comb tooth portion, and the comb tooth portion includes multiple comb teeth;
a laser irradiation portion disposed on the brush portion to be adjacent to the comb tooth portion, wherein the laser irradiation portion irradiates a laser beam to a person's scalp;
a vibration portion disposed on the brush portion; and
a controlling portion disposed on the main body and electrically connected to the laser irradiation portion and the vibration portion to activates the operations of the laser irradiation portion and the vibration portion.

2. The comb according to claim 1, further comprising a static electricity protection portion disposed on the main body and electrically connected to the controlling portion.

3. The comb according to claim 2, wherein the static electricity protection portion is a negative ion irradiation circuit or a contact static electricity protection circuit.

4. The comb according to claim 1, further comprising a pivot portion connected between the brush portion and the handle portion, so that the brush portion and the handle portion are foldable against each other.

5. The comb according to claim 3, wherein the laser irradiation portion includes a plurality of laser emitters, the plurality of laser emitters are surface-adhesive laser diodes, and disposed at positions between any two adjacent rows of the comb teeth.

6. The comb according to claim 1, wherein the controlling portion includes a processing circuit and an input circuit, the processing circuit is electrically connected to the input circuit and correspondingly controls the laser irradiation portion and the vibration portion according to an input signal of the input circuit, wherein when the input signal is a first input signal, the processing circuit turns on the laser irradiation portion, and wherein when the input signal is a second input signal, the processing circuit activates the laser irradiation portion and the vibration portion.

7. The comb according to claim 6, wherein the input circuit is a dip switch or a push-button switch.

8. The comb according to claim 1, wherein when the controlling portion activates the laser irradiation portion, the controlling portion outputs a prompt signal at intervals to prompt a user to move the comb.

9. The comb according to claim 8, wherein the prompt signal is a vibration signal outputted from the vibration portion or an audio signal outputted from a buzzer, wherein the buzzer is electrically connected to and controlled by the controlling portion.

10. The comb according to claim 1, further comprising:
a display portion disposed on the main body and electrically connected to the controlling portion, wherein the display portion correspondingly displays an operation message according to the control of the controlling portion, wherein the operation message includes at least a using time of the comb, a power state of the main body, or a working state of the laser irradiation portion;
a light indication portion electrically connected to and controlled by the controlling portion, wherein a light indication signal is outputted by the light indication portion to reflect a power charging state of the main body; and
a distance detection portion electrically connected to the controlling portion and disposed on the main body to detect a distance between the comb and an object, wherein when the distance between the comb and the object is smaller than a predetermined distance, the laser irradiation portion is activated to operate.
